(19) United States
(12) Patent Application Publication
(10) Pub. No.: US 2008/0206348 A1
(43) Pub. Date: Aug. 28, 2008

(54) Cilostazol-Containing Pharmaceutical Composition Based On Particles Of Less Than 50 Micrometers

(76) Inventors: Deepak Murpani, New Delhi (IN); Ashima Bhatti, Haryana (IN)

Correspondence Address: RANBAXY INC., 600 COLLEGE ROAD EAST, SUITE 2100, PRINCETON, NJ, 08540, US

(21) Appl. No.: 11/575,000

(22) PCT Filed: Sep. 16, 2005
(86) PCT No.: PCT/IB05/02750

§ 371 Date: Nov. 5, 2007

(30) Foreign Application Priority Data
Sep. 17, 2004 (IN) 1770/DEL/2004

(51) Int. Cl. A61K 31/4709; A61K 9/14; A61P 1/04; A61P 29/00; A61P 9/12

(52) U.S. Cl. 424/499; 514/312; 424/489

(57) ABSTRACT

The present invention relates to cilostazol compositions, process for their preparation, and methods for their administration to treat a condition. In the cilostazol compositions, 90% of the cilostazol particles have a particle size less than about 50 μm.

### FIELD OF THE INVENTION

The technical field of the present invention relates to cilostazol compositions, process for their preparation, and methods for their administration to treat a condition. In the cilostazol compositions, 90% of the cilostazol particles have a particle size less than about 50 μm.

### BACKGROUND OF THE INVENTION

Cilostazol, described in U.S. Pat. No. 4,277,479, is commercially available in 50 and 100 mg oral tablet strengths that are marketed by Otsuka under the trade mane PLETAL®. This drug shows a high platelet aggregation suppression action as well as various other types of medical actions, such as a phosphodiesterase inhibition action, an anti-ulcer action, a hypotensive action and an anti-phlogistic action. Cilostazol is practically insoluble in water and thus results in poor bioavailability of the drug.

Low solubility drugs often exhibit the drawback of insufficient dissolution in gastric fluids that reduces the likelihood of obtaining a plasmatic concentration sufficient to achieve the desired therapeutic effects. Much work has been undertaken worldwide on poorly soluble drugs to achieve a sufficient plasmatic concentration necessary to reach the desired therapeutic effects. Some of the frequently used approaches involve one or more of milling, solid dispersion, solid-state modifications, complexation, etc.

Cilostazol is a low solubility drug and requires well-defined solubility improving approaches to achieve the desired dissolution profile from cilostazol compositions. US Patent Application No. 20030166937 discloses pharmaceutical compositions of cilostazol that include cilostazol in which 90% of the particles either have a diameter of about 60 μm or less than about 15 μm.

Therefore, there is a need for cilostazol compositions that are bioequivalent when compared to the existing marketed compositions, such as PLETAL®, and are not only easier to manufacture, but also have a reproducible performance when compared to the existing compositions. None of the above prior art documents are believed to teach or suggest the present invention.

We have now discovered that cilostazol compositions having the desired dissolution profile may be prepared by using milled cilostazol wherein, at least 90% of the cilostazol particles have a particle size less than about 50 μm. The desired particle size may be achieved by processing cilostazol alone, or with a carrier to form a co-milled mass.

### SUMMARY OF THE INVENTION

In one general aspect there is provided a pharmaceutical composition that includes cilostazol particles. In the composition, at least 90% of the cilostazol particles are less than about 50 μm.

Embodiments of the composition may include one or more of the following features. For example, at least 90% of the cilostazol particles may be less than about 50 μm and at least 25% of the cilostazol particles may be greater than about 15 μm. At least 90% of the cilostazol particles may be less than about 45 μm and at least 50% cilostazol particles may be greater than about 15 μm.

The cilostazol particles may be prepared by a process of milling. The cilostazol may be blended with a carrier and milled to form a co-milled mass of the particle size. The carrier may include one or more of cellulose derivatives, sugars and starch. A weight ratio of the cilostazol and the carrier may be from about 1:1 to about 1:0.1

The compositions may include one or more pharmaceutically inert excipients including one or more of binders, diluents, surfactants, lubricants/glidants, and coloring agents.

In another general aspect there is provided a pharmaceutical composition that includes cilostazol particles. At least 25% of the cilostazol particles are greater than about 15 μm.

Embodiments of the pharmaceutical composition may include one or more of the following features. For example, at least 90% of the cilostazol particles may be less than about 50 μm and at least 25% cilostazol particles may be greater than about 15 μm. At least 90% of the cilostazol particles may be less than about 45 μm and at least 50% cilostazol particles may be greater than about 15 μm.

The cilostazol particles may be prepared by the process of milling. The cilostazol may be blended with a carrier and milled to form a co-milled mass of the particle size. The carrier may be one or more of cellulose derivatives, sugars and starch. A weight ratio of the cilostazol and the carrier may be from about 1:1 to about 1:0.1.

The pharmaceutical compositions may further include one or more pharmaceutically inert excipients including binders, diluents, surfactants, lubricants/glidants, and coloring agents.

In another general aspect there is provided a process for the preparation of a pharmaceutical composition of cilostazol particles. The process includes the steps of blending milled cilostazol particles or a co-milled mass of cilostazol particles with one or more pharmaceutically inert excipients; and processing the blend into a solid dosage form. At least 90% the cilostazol particles or co-milled mass of cilostazol particles are less than about 50 μm.

Embodiments of the process may include one or more of the following features. For example, the milling may include one or more of air jet milling, mechanical milling, cad milling, fitz milling, multi milling, impact milling, and ball milling. At least 90% of the cilostazol particles or co-milled mass of cilostazol particles may be less than about 50 μm and at least 25% of the cilostazol particles may be greater than about 15 μm.

The process may further include granulating the blend and the granulation includes wet granulation or dry granulation.

In another general aspect there is provided a process for the preparation of a pharmaceutical composition of cilostazol particles. The process includes the steps of blending milled cilostazol particles or a co-milled mass of cilostazol particles with one or more pharmaceutically inert excipients, and processing into a solid dosage form. At least 25% of the cilostazol particles or co-milled mass of cilostazol particles are greater than about 15 μm.

Embodiments of the process may include one or more of the following features. For example, the milling may include one or more of air jet milling, mechanical milling, cad milling, fitz milling, multi milling, impact milling, and ball milling. At least 90% of the cilostazol particles or co-milled mass of cilostazol particles may be less than about 50 μm and at least 25% cilostazol particles may be greater than about 15 μm.

The process may further include granulating the blend and the granulation includes wet granulation or dry granulation.

In another general aspect there is provided a method of treating one or more of ulcer, inflammation, hypertension and conditions associated with increased phosphodiesterase in mammals in need of such treatment. The method includes administering a pharmaceutical composition a pharmaceutical composition that includes cilostazol particles. In the composition, at least 90% of the cilostazol particles are less than about 50 μm. Embodiments of the method may include any of the features described above.

In another general aspect, there is provided a method of treating one or more of ulcer, inflammation, hypertension and conditions associated with increased phosphodiesterase in mammals in need of such treatment. The method includes administering a pharmaceutical composition that includes cilostazol particles. In the composition, at least 25% of the cilostazol particles are greater than about 15 μm. Embodiments of the method may include any of the features described above.

The details of one or more embodiments are set forth in the description below. Other features, objects and advantages of the invention will be apparent from the description and claims.

### DETAILED DESCRIPTION OF THE INVENTION

The present inventions are not limited to particular process steps and materials disclosed herein, but are extended to equivalents thereof as would be recognized by those of ordinary skill in the relevant arts. It should also be understood that terminology employed herein is used for the purpose of describing particular embodiments only and is not intended to be limiting.

The term “solid dosage form” as used herein includes conventional solid dosage forms such as tablet, capsule, granules, sachet, and the like.

The term “particle size” as used herein refers to the average particle diameter of the particle on conversion of its volume into a sphere. The percentage of cilostazol or co-milled mass particles as used herein refers to percentage volume of the total volume. The size of particles was measured using Malvern Mastersizer.

The term “cilostazol” as used herein includes cilostazol and its pharmaceutically acceptable salts.

Cilostazol is poorly soluble in water and its bioavailability is limited by the rate of dissolution of cilostazol into the surrounding media. Reduction of particle size results in an increase in the effective exposed surface to the dissolution media, aiding in solubility and consequently the bioavailability of cilostazol from the dosage form. However, uncontrolled size reduction to a very fine range may result in excessive drug losses during processing, besides hindering smooth processing of dosage forms. In addition, use of fine particles could also increase the risk of re-agglomeration.

To avoid such processing hurdles the present invention relates to pharmaceutical compositions comprising cilostazol particles of particular size ranges, i.e., at least 90% of the cilostazol particles have a particle size less than about 50 μm and/or at least 25% of the cilostazol particles have a particle size greater than about 15 μm. In particular, at least 90% of the cilostazol particles have a particle size less than about 45 μm and at least 25% have a particle size greater than about 15 μm. More particularly, at least 90% of the cilostazol particles have a particle size less than about 45 μm and at least 50% have a particle size greater than about 15 μm.

Cilostazol particles of the desired size range may be obtained by the process of milling using an air jet mill or any conventionally used mechanical mill, such as cad mill, fitz mill, multi mill, impact mill, and ball mill. In particular, a mechanical mill may be used. The particle size of the final product in a mechanical mill is dependent on the speed of rotation, aperture size and shape, and configuration of the screen/sieve used. These parameters can be easily adjusted/selected and maintained throughout the milling process, and thereby these mills can be expected to produce reproducible results. Advantageously, the particle size of the final product is not drastically affected by the initial particle size distribution and feed rate. These ensure reproducible particle size distribution of the final product, lowering variability in dissolution and consequently the bioavailability. Above all, mechanical milling is a fast and commercially feasible process.

Alternatively, cilostazol may be blended with a carrier and then milled to form a co-milled mass. During co-milling cilostazol particles may adhere to the carrier surface in a fine particulate form. By virtue of such adherence, static charges generated during the milling process are neutralized, thereby reducing chances of re-agglomeration of cilostazol particles besides improving the flow properties. Co-milling also helps in wetting of cilostazol during dissolution, enhancing dissolution further. In embodiments wherein the term “co-milled mass” is used, the particle sizes and respective percentages of cilostazol, as provided above, refers to the particles of the co-milled mass.

Examples of carriers used for co-milling include all substances that are physiologically acceptable, compatible with cilostazol and other pharmaceutically inert excipients, and have a capacity to adhere to cilostazol particles. Specific examples include cellulose derivatives such as microcrystalline cellulose and calcium carboxymethylcellulose; sugars such as lactose; and starch. The weight ratio of cilostazol and carrier may vary from about 1:1 to about 1:0.1. In particular, starch may be used in a weight ratio of 1:0.3 (cilostazol:starch).

According to one of the embodiments, pharmaceutical compositions of cilostazol may comprise milled cilostazol, or a co-milled mass, and one or more pharmaceutically inert excipients.

Pharmaceutically inert excipients include all physiologically inert excipients used in the pharmaceutical art of dispensing. Examples include binders, diluents, surfactants, disintegrants, lubricants/glidants, coloring agents, and the like.

Specific examples of binders include methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate, propylene glycol, and the like.

Specific examples of diluents include calcium carbonate, calcium phosphate-dibasic, calcium phosphate-tribasic, calcium sulfate, microcrystalline cellulose, cellulose powdered, dextrates, dextrins, dextrose excipients, fructose, kaolin, lactitol, lactose, mannitol, sorbitol, starch, starch pregelatinized, sucrose, sugar compressible, sugar confectioners, and the like and mixtures thereof.

Surfactants include both non-ionic and ionic (cationic, anionic and zwitterionic) surfactants suitable for use in pharmaceutical dosage forms. These include polyethoxylated fatty acids and its derivatives, for example, polyethylene glycol 400 distearate, polyethylene glycol-20 dioleate, polyethylene glycol 4-150 mono dilaurate, and polyethylene glycol-20 glyceryl stearate; alcohol-oil transesterification products, for example, polyethylene glycol-6 corn oil; polyglycerized fatty acids, for example, polyglyceryl-6 pentaoleate; propylene glycol fatty acid esters, for example, propylene glycol monocaprylate; mono and diglycerides, for example, glyceryl ricinoleate; sterol and sterol derivatives; sorbitan fatty acid esters and its derivatives, for example, polyethylene glycol-20 sorbitan monooleate and sorbitan monolaurate; polyethylene glycol alkyl ether or phenols, for example, polyethylene glycol-20 cetyl ether and polyethylene glycol-10-100 nonyl phenol; sugar esters, for example, sucrose monopalmitate; polyoxyethylene-polyoxypropylene block copolymers known as “poloxamer”; ionic surfactants, for example, sodium caproate, sodium glycocholate, soy lecithin, sodium stearyl fumarate, propylene glycol alginate, octyl sulfosuccinate disodium, and palmitoyl carnitine; and the like and mixtures thereof.

Specific examples of disintegrants include low-substituted hydroxypropylcellulose L-HPC), sodium starch glycollate, carboxymethyl cellulose, calcium carboxymethyl cellulose, sodium carboxymethyl cellulose, croscarmellose sodium A-type (Ac-di-sol), starch, crystalline cellulose, hydroxypropyl starch, pregelatinized starch, and the like and mixtures thereof.

Specific examples of lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, and the like and mixtures thereof.

Coloring agents include any FDA approved colors for oral use.

In one of the embodiments, cilostazol tablets may be prepared by blending milled cilostazol, or a co-milled mass of cilostazol, and a carrier with one or more pharmaceutically inert excipients and directly compressing the blend into tablets.

In another embodiment, cilostazol tablets may be prepared by blending milled cilostazol, or a co-milled mass of cilostazol, and a carrier with one or more intragranular pharmaceutically inert excipients; wet granulating the blend with a granulating fluid or solution/dispersion of binder in granulating fluid; drying and sizing the granules; blending with extragranular pharmaceutically inert excipients; lubricating the blend; and compressing the blend into tablets.

In another embodiment, cilostazol tablet may be prepared by blending milled cilostazol, or a co-milled mass of cilostazol, and a carrier with one or more intragranular pharmaceutically inert excipients; dry granulating the blend by roller compaction or slugging; sizing the granules; blending with extragranular excipients; lubricating the blend; and compressing the blend into tablets.

Specific examples of solvents used as the granulating fluid and for preparing a solution/dispersion of binder include methylene chloride, isopropyl alcohol, acetone, methanol, ethanol, water and the like.

Optionally, tablets prepared in any of the embodiments described above may be further coated with one or more functional and/or non-functional coating layers as desired.

The invention is further illustrated by the following examples, which are for illustrative purposes only and should not be construed as limiting the scope of the inventions in any way.

### EXAMPLES

Cilostazol Tablet Composition (Examples 1 and 2)

| Ingredient | Weight (mg)/tablet |
|---|---|
| Cilostazol | 100.0 |
| Starch | 30.0 |
| Hydroxypropyl methylcellulose (5 cps) | 1.5 |
| Calcium carboxymethylcellulose | 12.0 |
| Microcrystalline cellulose | 24.5 |
| Magnesium stearate | 1.5 |
| Purified water | q.s. |

### Procedure (Example 1)

1. Cilostazol was sifted through sieve #25 (BSS) and milled in a Fitz mill such that 90% of the particles were less than 44 μm and 30% were greater than 15 μm.

2. Starch, calcium carboxymethylcellulose, hydroxypropyl methylcellulose and microcrystalline cellulose were each sifted separately through sieve #25 (BSS).

3. The sifted calcium carboxymethylcellulose, hydroxypropyl methylcellulose, microcrystalline cellulose, and half of the quantity of starch of step 2 were blended with the milled particles of step 1 in a rotary mixer granulator to form a blend.

4. The blend of step 3 was granulated using purified water as granulating fluid to form granules.

5. The granules of step 4 were dried in a fluidized bed dryer and sized by sifting through sieve #25 (BSS).

6. Sized granules of step 5 were blended with the remaining half quantity of starch (of step 2) and magnesium stearate, and compressed into tablets using suitable toolings.

### Procedure (Example 2)

1. Cilostazol and starch were sifted through sieve #25 (BSS) and co-milled in a Fitz mill such that 90% of the particles were less than 44 μm.

2. Calcium carboxymethylcellulose, hydroxypropyl methylcellulose and microcrystalline cellulose were sifted separately through sieve #25 (BSS).

3. The sifted ingredients of step 2 were blended with the co-milled mass of step 1 in a rotary mixer granulator to form a blend.

4. The blend of step 3 was granulated using purified water as the granulating fluid to form granules.

5. The granules of step 4 were dried in a fluidized bed dryer and sized by sifting through sieve #25 (BSS).

6. The sized granules of step 5 were blended with magnesium stearate and compressed into tablets using suitable toolings.

The comparative in vitro release of cilostazol from tablets prepared according to the procedure and compositions of Examples 1 and 2, and the marketed Pletal® (100 mg) tablets was studied in 900 ml water containing 0.3% sodium lauryl sulphate, using USP II dissolution apparatus, at a paddle speed of 75 rpm. The results of the study are given in Table 1.

TABLE 1
In vitro release of cilostazol from Pletal ® tablets and tablets
prepared according to Examples 1 and 2

| | Cumulative percentage (%) | | |
|---|---|---|---|
| | release of cilostazol | | |
| Time (Mn) | Example 1 | Example 2 | Pletal ® tablets |
| 5 | 31 | 39 | 44 |
| 15 | 65 | 63 | 75 |
| 30 | 77 | 77 | 86 |
| 45 | 86 | 84 | 92 |
| 60 | 89 | 89 | 95 |

As illustrated in Table 1, the cumulative percentage release of cilostazol from the tablets of Examples 1 and 2 are similar to the cumulative percentage release of the Pletal® tablets.

While several particular forms of the inventions have been described, it will be apparent that various modifications and combinations of the inventions detailed in the text can be made without departing from the spirit and scope of the inventions. Accordingly, it is not intended that the inventions be limited, except as by the appended claims.

We claim:

1. A pharmaceutical composition comprising cilostazol particles, wherein at least 90% of the cilostazol particles are less than about 50 μm.

2. The pharmaceutical composition according to claim 1, wherein at least 90% of the cilostazol particles are less than about 50 μm and at least 25% of the cilostazol particles are greater than about 15 μm.

3. The pharmaceutical composition according to claim 2, wherein at least 90% of the cilostazol particles are less than about 45 μm and at least 50% cilostazol particles are greater than about 15 μm.

4. The pharmaceutical composition according to claim 1, wherein the cilostazol particles are prepared by a process of milling.

5. The pharmaceutical composition according to claim 1, wherein the cilostazol is blended with a carrier and milled to form a co-milled mass of the particle size.

6. The pharmaceutical composition according to claim 5, wherein the carrier comprises one or more of cellulose derivatives, sugars and starch.

7. The pharmaceutical composition according to claim 6, wherein a weight ratio of the cilostazol and the carrier comprises from about 1:1 to about 1:0.1

8. The pharmaceutical composition according to claim 1, wherein the compositions further comprises one or more pharmaceutically inert excipients comprising one or more of binders, diluents, surfactants, lubricants/glidants, and coloring agents.

9. A pharmaceutical composition comprising cilostazol particles, wherein at least 25% of the cilostazol particles are greater than about 15 μm.

10. The pharmaceutical composition according to claim 9, wherein at least 90% of the cilostazol particles are less than about 50 μm and at least 25% cilostazol particles are greater than about 15 μm.

11. The pharmaceutical composition according to claim 9, wherein at least 90% of the cilostazol particles are less than about 45 μm and at least 50% cilostazol particles are greater than about 15 μm.

12. The pharmaceutical composition according claim 9, wherein the cilostazol particles are prepared by the process of milling.

13. The pharmaceutical composition according to claim 9, wherein the cilostazol is blended with a carrier and milled to form a co-milled mass of the particle size.

14. The pharmaceutical composition according to claim 13, wherein the carrier one or more of cellulose derivatives, sugars and starch.

15. The pharmaceutical composition according claim 13, wherein a weight ratio of the cilostazol and the carrier comprises from about 1:1 to about 1:0.1

16. The pharmaceutical composition according to claim 9, wherein the pharmaceutical compositions further comprises one or more pharmaceutically inert excipients comprising binders, diluents, surfactants, lubricants/glidants, and coloring agents.

17. A process for the preparation of a pharmaceutical composition of cilostazol particles comprising the steps of:
blending milled cilostazol particles or a co-milled mass of cilostazol particles with one or more pharmaceutically inert excipients; and
(b) processing the blend into a solid dosage form,
wherein at least 90% the cilostazol particles or co-milled mass of cilostazol particles are less than about 50 μm.

18. The process according to claim 17, wherein the milling comprises one or more of air jet milling, mechanical milling, cad milling, fitz milling, multi milling, impact milling, and ball milling.

19. The process according to claims 17, wherein at least 90% of the cilostazol particles or co-milled mass of cilostazol particles are less than about 50 μm and at least 25% of the cilostazol particles are greater than about 15 μm.

20. The process according to claim 17, further comprising granulating the blend of step (a), wherein the granulation comprises wet granulation or dry granulation.

21. A process for the preparation of a pharmaceutical composition of cilostazol particles comprising the steps of:
(a) blending milled cilostazol particles or a co-milled mass of cilostazol particles with one or more pharmaceutically inert excipients; and
(b) processing into a solid dosage form,
wherein at least 25% of the cilostazol particles or co-milled mass of cilostazol particles are greater than about 15 μm.

22. The process according to claim 21, wherein the milling comprises one or more of air jet milling, mechanical milling, cad milling, fitz milling, multi milling, impact milling, and ball milling.

23. The process according to claim 21, wherein at least 90% of the cilostazol particles or co-milled mass of cilostazol particles are less than about 50 μm and at least 25% cilostazol particles are greater than about 15 μm.

24. The process according to claim 21, further comprising granulating the blend of step (a), wherein the granulation comprises wet granulation or dry granulation.

25. A method of treating one or more of ulcer, inflammation, hypertension and conditions associated with increased phosphodiesterase in mammals in need of such treatment, the method comprising administering a pharmaceutical composition of cilostazol according to claim 1.

26. A method of treating one or more of ulcer, inflammation, hypertension and conditions associated with increased phosphodiesterase in mammals in need of such treatment, the method comprising administering a pharmaceutical composition of cilostazol according to claim 2.
